(19) United States
(12) Patent Application Publication
(10) Pub. No.: US 2002/0172712 A1
(43) Pub. Date: Nov. 21, 2002

(54) Antiemetic, anti-motion sustained release drug delivery system

(76) Inventors: Alan Drizen, Downsview (CA); Gary M. Nath, Bethesda, MD (US)

Correspondence Address: NATH & ASSOCIATES PLLC, 6th Floor, 1030th 15th Street, N.W., Washington, DC, 20005, US

(21) Appl. No.: 09/810,329

(22) Filed: Mar. 19, 2001

(51) Int. Cl. A61K 9/70

(52) U.S. Cl. 424/486

(57) ABSTRACT

This invention relates to a stable, sterilized, purified composition having a polymer matrix and a therapeutically effective amount of a drug, wherein the drug can be used to prevent or treat drug-induced, alcohol-induced, biologically-induced, trauma-induced or pain-induced nausea, vomiting, dizziness and other adverse effects arising from but not limited to motion sickness, cancer therapy, and pregnancy. In particular, the polymer matrix may be conformable to topical application on animal skin.

### BACKGROUND OF THE INVENTION

1. Field of the Invention

This invention relates to a dermal dressing for conformable topical application and sustained release of a polymer matrix containing a drug or combinations of drugs to animal skin. The drug can be any pharmaceutically effective amount useful for preventing and treating nausea, vomiting, dizziness and other adverse effects arising from but not limited to motion sickness, cancer therapy, and pregnancy in an animal.

2. Description of the Related Art

Over the years, methods have been developed to achieve the efficient delivery of a therapeutic drug to a mammalian body part requiring pharmaceutical treatment. Intravenous delivery and oral ingestion are two examples of current delivery techniques. While these techniques are generally effective, they suffer from several pharmacokinetic limitations and often result in substantial non-compliance by patients. For example, the therapeutic benefit from conventional methods often wear off within several hours after the initial dosing while the pain and discomfort associated with injections and intravenous lines often lead to difficulties in administration and maintenance of intravenous lines. Even oral administration can be ineffective where a patient cannot ingest due to nausea and/or vomiting.

Topical administration of a pharmaceutically effective agent may avoid the problems associated with known drug delivery methods. One known method of topical administration uses an aqueous liquid that is applied at room temperature but forms a semi-solid gel when warmed to body temperature. This technique has the reported benefit of being easier to use and improving drug retention at the treatment site. For example, U.S. Pat. No. 4,188,373 uses PLURONIC® polyols in aqueous compositions to thermally gel aqueous systems. A sol-gel transition temperature is adjusted by varying the concentration of the polyols.

U.S. Pat. Nos. 4,474,751 and 4,478,822 also disclose drug delivery systems utilizing thermosetting gels. Here, gel transition temperature and/or gel rigidity is modified by adjusting the pH, ionic strength, and the concentration of the polymer. Other patents disclosing aqueous gel compositions are U.S. Pat. Nos. 4,883,660; 4,767,619; 4,511,563; 4,861,760; and 5,318,780. U.S. Pat. No. 4,911,926 discloses a thermosetting gel for the treatment of injured mammalian tissue of the thoracic or peritoneal cavities.

Thermosetting gels can also use a matrix to deliver a drug. Bodmeier et al., disclose in Volume 78, Number 11, of the November 1989 issue of the Journal of Pharmaceutical Science, ionic polysaccharides such as chitosan or sodium alginate that form spherical agglomerates of water-insoluble drugs into a matrix for drug delivery. Calcium alginate gel formulations can also be used as a matrix material, as disclosed in the Journal of Controlled Release,(1986), pages 229-233, Pfister et al.

U.S. Pat. No. 3,640,741, takes another approach to gelling a compound by using cross-linkers. For example, a molded plastic mass composed of a hydrophilic colloid such as carboxymethyl cellulose gum or a natural alginate gum is suspended in an organic liquid medium such as glycerin with a cross-linking agent such as a liquid polyol. The hydrophilic colloid can then be cross-linked with a polyol by accelerating the cross-linking reaction with aluminum and calcium salts. Another useful colloid is chitosan as disclosed in U.S. Pat. No. 4,895,724. Chitosan can be cross-linked utilizing aldehydes, epichlorohydrin and benzoquinone.

Another method of drug delivery using aqueous compositions is disclosed in U.S. Pat. No. 4,795,642, whereby gelatin-encapsulated capsules enclose a solid matrix. The matrix is formed by cation-assisted gelation of a liquid-filling composition incorporating a vegetable gum in combination with a pharmaceutically-active compound. The vegetable gum can be a polysaccharide gum such as an alginate.

While osmotic drug delivery systems are disclosed in the prior art, the isotonicity of the aqueous drug delivery vehicles are never contemplated. For example U.S. Pat. No. 4,439,196 only discloses a multi-chamber compartment for holding osmotic agents, adjuvants, enzymes, drugs, pro-drugs, pesticides, and the like. These materials are enclosed by a semipermeable membrane where the membrane is contacted with the intended target thereby allowing the fluids within the chambers to diffuse across the membrane. However, the prior art pharmaceutical preparations are not isotonic with mammalian blood because the disclosed device relies on the permeability of the membrane to control the rate of delivery.

To date, the United States generally disapproves the sale of prescription medications formulated for topical administration. Despite the apparent advantages of topical administration, proof of their ability to transdermally transport a drug through the skin has been heretofore been nonexistent. Blood and urine samples of patients treated with prior art topically treatments consistently fail to show appreciable amount of drug. Although topically applied counter-irritants such as menthol, eucalyptus, and camphor are approved for sale over the counter, these products are designed to treat only minor problems. Moreover, counter-irritation does not require the type of deep penetration of tissue structures required by pharmaceuticals. In short, no current prior art method, other than those developed by inventors hereto, exists for the deep treatment of major maladies through topical application.

Other known methods of topical drug delivery attempt to overcome the deficiencies of the prior art by increasing adsorption through the use of bandages and dressings.

U.S. Pat. No. 5,814,031 discloses a structured wound dressing comprising an adhesive bandage containing a hydrophobic solvent, a network polymer and a flow control agent useful in healing wounds.

U.S. Pat. No. 5,415,866, on the other hand, discloses a drug delivery system for the topical administration of medication comprising a medicated gel pad encapsulated between two layers of liquid fraction impermeable material. The medicated pad contacts the skin through a drug delivery aperature formed in the liquid fraction layer touching the skin. The layer contacting the skin contains a pressure sensitive adhesive to keep the pad on the skin.

In contrast, U.S. Pat. No. 5,538,500 keeps the pad on the skin with an adhesive. U.S. Pat. No. 5,538,500 discloses a medical dressing comprising an elastic bandage wrap, an absorbent pad affixed to the wrap and a medicated gauze affixed to the absorbent pad. An adhesive is affixed to the perimeter of the gauze thereby adhering to the wearer's skin.

A site specific application is disclosed in U.S. Pat. No. 5,662,925, which discloses a device for administering an active agent to skin or mucosa. The device comprises a laminated composite of adhesive overlay, a mounting layer underlying the center of the adhesive overlay and a membrane permeable to the active agent. The membrane and the backing layer form a reservoir that contains the active agent. A peel seal disc underlies the permeable membrane and a heat seal is set about the periphery of the peel seal disc. The peel seal disc protects against release of the active agent from the reservoir and the heat seal protects the active agent from exposure to the environment prior to use.

U.S. Pat. No. 6,086,912 discloses a drug delivery system for the topical administration of medication or other therapeutic material. The medication is contained in a reservoir formed by the inner surface of a backing film and a permeable micro-porous membrane. The reservoir is sealed by a disc composed of several layers of opaque material. A release liner is attached to the disc. Instead of an adhesive layer, the medication is held against the skin by a bandage around the patient's body part.

Despite the prior art's mechanical facilitation of adsorption, chemical limitations persist. One such chemical limitation is the failure to adequately deliver chemicals through the outer skin layer.

It has been discovered that hyaluronic acid may be effective in the treatment of skin injuries, wounds and other conditions. Hyaluronic acid is a naturally occurring polysaccharide containing alternating D-glucoronic acid monosaccharide units (GlcUA) and N-acetyl-D-glucosamine (GlcNAc) linked by glycosidic bonds which are alternately linked with 1→3 and 1→4 glycoside bonds. Thus the formula for the repeating unit is (1→3)-β-GlcNAc-(1→4)=β-GlcUA. Hyaluronic acid generally possesses a linear copolymer structure of approximately 2,500 repeating disaccharide units and has an average molecular weight generally within the range of 50,000 to 8×10⁶ daltons or higher.

The biological importance of hyaluronic acid is demonstrated by the highly conserved nature of hyaluronic acid production genes in the evolutionary tree. These genes show little variation, generation to generation, and species to species. The highly conserved nature of the hyaluronic acid genome may be traced, in part, to the fact that hyaluronic acid is a major carbohydrate component of the extra-cellular matrix and can be found in the skin, eyes, and most other tissues and organs throughout the body. Additionally, extracellular hyaluronic acid has unique hygroscopic, rheological, and viscoelastic properties. For example, hyaluronic acid and its salts give rise to viscous, elastic solutions in water and physiological salt solutions.

Sodium hyaluronate also binds to many other extracellular matrix molecules through complex interaction with matrix components. Moreover, hyaluronic acid usually does not trigger the immune response cascade because hyaluronic acid is present in every living organism as an identical composition. Thus, hyaluronic acid is amenable to advanced medical uses, and as a consequence, it has been the subject of many modification attempts. For example, a 1% solution of sodium hyaluronate (Healon®) was described for use in eye viscosurgery. L. A. Pope and E. A. Balazs, Opthalmology, 87, No. 8, 1980.

Hyaluronic acid can also be used to improve or increase biocompatibility with other substances as disclosed in U.S. Pat. No. 4,500,676. Further, sodium hyaluronate has been shown to have a critical function in skin wound healing, based in part on the finding that the levels of sodium hyaluronate are temporarily elevated in granulation tissues.

Due to its unusual activity, a significant body of research conducted has been directed towards hyaluronic acid extraction from human and other animal tissue sources, as well as purification of hyaluronic acid products.

For example, U.S. Pat. No. 4,851,521 discloses total and partial esters of hyaluronic acid and their salts, as well as their preparation.

U.S. Pat. No. 4,782,046 discloses a protein and nucleic acid free hyaluronic acid composition prepared from a hyaluronidase-negative or hyaluronidase inhibited microbiological source.

U.S. Pat. No. 6,020,484 discloses a process for preparing a fraction of a hyaluronic acid by treating a hyaluronic acid, while in the presence of sodium hypochlorite, with ultrasound.

U.S. Pat. No. 5,403,592 discloses an injectable, lubricating composition for joint pain consisting essentially of at least one surface active phospholipid and hyaluronic acid.

U.S. Pat. No. 5,876,744 discloses highly bioadhesive and mucoadhesive aqueous compositions and processes for preparation thereof, useful for rehydration of the skin and mucosal tissues and suitable as a vehicle in percutaneous absorption.

Research has further focused on the high-biocompatibility of hyaluronic acid, and numerous references describe combining the acid with drugs for transdermal drug delivery.

U.S. Pat. No. 5,128,326 discloses a drug delivery system comprising an insoluble hyaluronan or soluble hylan copolymerized with at least one more substance having biological or pharmacological activity which is controllably released from said system.

U.S. Pat. No. 5,728,391 discloses a process for treating skin disease consisting of xerosis senilis, asteatosis, keratodermia tylodes palmaris progressive, keratosis palmaris et plantaris, ichthyosis, lichen pilaris, pityriasis rosea Gilbert, and milaria, by topically applying an effective amount of hyaluronic acid.

Pat. No. 5,624,915 discloses a method for treating eczema by applying a therapeutically effective amount of a composition comprising a pharmaceutically acceptable carrier, urea, and hyaluronic acid.

U.S. Pat. No. 5,733,891 discloses a compound consisting of a covalently bonded anti-cancer agent and hyaluronic acid.

U.S. Pat. No. 5,824,658 discloses a method for treating pain topically using a non-steroidal anti-inflammatory drug, a form of hyaluronic acid, or a combination thereof.

U.S. Pat. No. 5,910,489 discloses a method of treating liver spots, malignancies of the skin, genital warts, cervical cancer, psoriasis, corns on the feet, and hair loss on the head of pregnant women by administering to the skin a pharmaceutical composition consisting of a form of hyaluronic acid.

U.S. Pat. No. 5,977,088 discloses a method of treating pain topically by administering to the skin a composition comprising a non-steroidal anti-inflammatory drug and a form of hyaluronic acid.

U.S. Pat. No. 5,985,850 discloses a pharmaceutical composition containing hyaluronic acid and an agent for the treatment of diseases and conditions relating to underperfused and pathological tissue.

U.S. Pat. No. 6,017,900 discloses a composition for topical administration to a site of trauma of skin or tissue comprising a drug for treatment and hyaluronic acid.

U.S. Pat. No. 6,019,989 discloses a skin treatment comprising a skin activator with a glycosaminoglycan production-accelerating effect.

U.S. Pat. No. 5,409,904 discloses an example of a viscous hyaluronic acid solution comprising a therapeutically effective amount of a viscous or viscoelastic material selected from the group consisting of hyaluronic acid, chondroitin sulfate, modified collagen, modified cellulose, and combinations thereof in a physiologically compatible salt solution.

Despite known applications of hyaluronic acid, it has been unexpectedly discovered that highly concentrated polymer matrix formed from a negatively charged polymer such as hyaluronic acid associated with a non-ionic polymer such as hydroxyethylcellulose facilitates a sustained release or extended release delivery of a drug. Moreover, an addition of a therapeutically effective amount of a drug allows efficient pharmaceutical treatment. Concentrations of greater than about 10% by weight of a polymer matrix containing hyaluronic acid associated with a non-ionic polymer have also been unexpectedly attainable.

In this regard, U.S. Pat. No. 5,927,937 discloses a process for preparing a cross-linked biocompatible polysaccharide gel composition by cross-linking a water soluble polysaccharide in at least two steps, wherein the cross-linking is discontinued before galation by sterically hindering the reaction. But again high methods for achieving high concentrations are not disclosed.

U.S. Pat. No. 5,783,691 also discloses an invention relating to the cross-linking hyaluronic acid derivatives by means of a reaction with a phosphorous containing reagent. The invention preparing a water-insoluble biocompatible gel comprising reacting hyaluronic acid with carbodiimide.

None of the known hyaluronic acid compositions or methods, however, are able to administer effective therapeutic amounts of a medicine or biologically active agent for sustained periods of time, i.e. longer than 1 to 24 hours, and preferably at least 8 hours, without repeated administration of the composition every 2 to 5 hours.

In addition, a more dense gel is required to give proper effect to the active ingredients. If a gel were merely compacted in volume, it would rapidly swell in animal tissues where there is free access to water.

Accordingly, a dermal adhesive dressing containing a polymer matrix containing a negatively charged polymer such as hyaluronic acid associated with a non-ionic polymer such as hydroxyethylcellulose which would allow for the transdermal administration a drug over a long period of time is needed. Additionally, the dermal dressing must provide for transdermal adsorption of a concentrated polymer matrix, alone or in combination with another drug, to the animal, such that the drug does not have to be administered again for at least 24 hours. Additionally, a method for concentrating the polymer matrix as well as a method of manufacturing and administering to heal or treat a condition in an animal is needed.

### SUMMARY OF THE INVENTION

The present invention relates to a stable, sterilized, purified composition containing a polymer matrix and a therapeutically effective amount of a drug, wherein the drug can be selected from the group of serotonin receptor antagonists, anti-dopaminergics, metclopramides, and scopolamine, dronabinol, ondansetron, granisetron, phenothiazine, thioridazine, diazepam, meclizine, ergoloid mesylates, chlorpromazine, trimethobenzamide, thiethylperazine, perphenazine, hydroxyzine pamoate, compazine, peragen, thorazine, tigan, or mixtures thereof, or wherein the drug can be selected from the group of chemotherapeutics such as actinomycin D, adriamycin, altretamine, asparaginase, bleomycin, busulphan, capecitabine, carboplatin, carmustine, chlorambucil, cisplatin, cyclophosphamide, cytarabine, dacarbazine, daunorubicin, doxorubicin, epirubicin, etoposide, fludarabine, fluorouracil, flutamide, gemcitabine, hydroxyurea, idarubicin, ifosfamide, interferon, irinotecan, leuprolide, liposomal doxorubicin, lomustine, megestrol, melphalan, mercaptopurine, methotrexate, mitomycin, mitozantrone, mechlorethamine oxaliplatin, procarbazine, steroids, streptozocin, taxol, taxotere, tamoxifen, tamozolomide, thioguanine, thiotepa, tomudex, topotecan, treosulfan, vinblastine, vincristine, vindesine, vinorelbine, buserelin, chlorotranisene, chromic phosphate, dexamethasone, estradiol, estradiol valerate, estrogens conjugated and esterified, estrone, ethinyl estradiol, floxuridine, goserelin, and prednisone, or mixtures thereof, or wherein the drug can be selected from the group of compounds useful for treating alcoholism such as benzodiazepines, barbiturates, librium, serax, tranxene, valium diazepam, lorazepam, oxazepam, and lorazepam, or mixtures thereof.

More particularly, the polymer matrix contains a negatively charged polymer in combination with a nonionic polymer wherein the nonionic polymer is selected from the group consisting of hydroxyethylcellulose, hydroxypropylcellulose, or carboxymethylcellulose and the negatively charged polymer is selected from the group of a hyaluronic acid, a hyaluronic acid salt and mixtures thereof.

Another embodiment of the present inventive subject matter is a dermal dressing containing a polymer matrix having a negatively charged polymer in combination with a nonionic polymer, wherein the polymer matrix is conformable to topical application on animal skin and where the polymer matrix contains a therapeutically effective amount of a drug. More particularly, the dermal dressing has a backing sheet having an adhesive capable of securing the dermal dressing to the animal skin, a reservoir affixed to the backing sheet and a porous membrane interposed between the polymer matrix and the animal skin.

Yet another embodiment of the present inventive subject matter is a dermal dressing having a backing sheet overlying a polymer matrix, wherein the backing sheet has an adhesive capable of securing the polymer matrix to the backing sheet and the backing sheet to animal skin, and a webbed covering layer underlying said polymer matrix.

Still yet another embodiment of the present inventive subject matter contemplates a dermal dressing having a support substrate overlying the backing sheet and the polymer matrix overlying the support substrate, wherein the backing sheet has an adhesive capable of securing the support substrate to the backing sheet and the backing sheet to animal skin.

Still yet another further embodiment of the present inventive subject matter contemplates a dermal dressing having a covering layer overlying the polymer matrix, one or more release sheets, wherein the backing sheet has applied thereto an adhesive which secures the support substrate to the backing sheet and the backing sheet to the animal skin, and wherein the release sheets completely cover the adhesive on the backing sheet and the covering layer, and wherein the release sheets may be peeled off of the adhesive.

Another embodiment of the present inventive subject matter contemplates a method for treating an animal, comprising the steps of applying a dermal dressing to animal skin, wherein the dermal dressing is comprised of a polymer matrix containing a negatively charged polymer in combination with a nonionic polymer, wherein the polymer matrix is conformable to topical application on animal skin, and wherein said polymer matrix contains a therapeutically effective amount of a drug.

Still another embodiment of the present inventive subject matter contemplates a method for preventing or treating a condition in an animal comprising the steps of applying a polymer matrix film onto the animal on an area to be treated, wherein the polymer matrix film contains a negatively charged polymer in combination with a nonionic polymer, and is formable, flexible and moveable, and wherein the polymer matrix film is secured to the polymer matrix film with a dressing fixative.

Still yet another embodiment of the present inventive subject matter contemplates a method for preventing or treating nausea in an animal for a sustained period of time, comprising the step of applying to the animal a polymer matrix, wherein the polymer matric contains a negatively charged polymer and a nonionic polymer in combination with a therapeutically effective amount of a drug for preventing or treating nausea.

Another embodiment of the present inventive subject matter contemplates a method for preventing or treating dizziness in an animal for a sustained period of time, comprising the step of applying to the animal a polymer matrix, comprising a negatively charged polymer and a nonionic polymer in combination with a therapeutically effective amount of a drug for preventing or treating dizziness.

Yet another embodiment of the present inventive subject matter contemplates a method for preventing or treating vomiting in an animal for a sustained period of time, comprising the step of applying to the animal a polymer matrix, comprising a negatively charged polymer and a nonionic polymer in combination with a therapeutically effective amount of a drug for preventing or treating vomiting.

Still yet another embodiment of the present inventive subject matter contemplates a method for preventing or treating pre-operative or post-operative vomiting, nausea or dizziness in an animal for a sustained period of time, comprising the step of applying to the animal a polymer matrix, comprising a negatively charged polymer and a nonionic polymer in combination with a therapeutically effective amount of a drug for preventing or treating pre-operative or post-operative vomiting, nausea or dizziness.

Still yet another further embodiment of the present inventive subject matter contemplates a method for preventing or treating cancer in an animal for a sustained period of time, comprising the step of applying to the animal a polymer matrix, comprising a negatively charged polymer and a nonionic polymer in combination with a therapeutically effective amount of a drug for preventing or treating cancer.

Another embodiment of the present inventive subject matter contemplates a method for preventing or treating alcohol-related disorders in an animal for a sustained period of time, comprising the step of applying to the animal a polymer matrix, comprising a negatively charged polymer and a nonionic polymer in combination with a therapeutically effective amount of a drug for preventing or treating alcohol-related disorders.

### BRIEF DESCRIPTION OF THE DRAWINGS

The invention may be better understood by reference to the drawings wherein:

FIG. 1 is a perspective view of a dressing, with portions cut away, of one embodiment of the present invention.

FIG. 2 is a perspective view of a dressing, with portions cut away, of another embodiment of the present invention (support substrate layer included).

FIG. 3 is an isometric view of FIG. 2, with release sheets in place.

FIG. 4 is a sectional view of FIG. 2, with release sheets in place.

FIG. 5 is a fragmentary view, greatly enhanced and partially in section, of a portion of the dressing as seen in FIG. 2.

FIG. 6 is a perspective view of a dressing, with portion cut away, of an embodiment of the present invention.

FIG. 7 is a sectional view of FIG. 6.

FIG. 8 is a schematic showing the manufacture of the dressing shown in FIG. 2.

### DETAILED DESCRIPTION OF THE INVENTION

The present invention relates to a dermal dressing as well as methods for manufacturing the dressing and using the dressing to prevent and treat conditions in an animal. The dressing of the present invention may be comprised of a polymer matrix or a separate dressing impregnated with the polymer matrix. The polymer matrix contains a negatively charged polymer such as hyaluronic acid associated with a non-ionic polymer such as hydroxyethylcellulose alone, in combination with other drugs, which can be topically administered to a patient in need thereof. The dressing of the present invention is able to occlusively cover the targeted area of the animal skin for treatment. This allows for better diffusion of the medication into the animal's skin.

Another benefit of the present invention is that a dosage of a therapeutically effective amount of a drug does not wear off within several hours of its application as is the case with conventional methods of drug delivery, such as a pill or intravenous injection. Because the drug in the dressing of the present invention is suspended in a specially designed polymer matrix containing almost equal molar ratios of a negatively charged polymer and a nonionic polymer suspended or dissolved in water, the drug only needs to be administered once over at least an hour to several day interval.

The polymer matrix is used to prevent or treat drug-induced, alcohol-induced, biologically-induced, trauma-induced or pain-induced nausea, vomiting, dizziness and other adverse effects arising from but not limited to motion sickness, cancer therapy, and pregnancy. The polymer matrix may also be used for cancer therapy, and may be administered alone to cancer patients or in combination with additional chemotherapeutic agents. Additionally, the matrix may be used to treat or prevent alcohol related disorders.

One embodiment of the dermal adhesive dressing of the present invention, comprises a backing sheet, a polymer matrix overlying the backing sheet and a webbed covering layer overlying the polymer matrix. The upper surface of the backing sheet is coated with an adhesive which secures the polymer matrix to the backing sheet and the backing sheet to the animal skin.

Another embodiment of the present invention is a dermal adhesive dressing, which comprises a backing sheet, a support substrate, a polymer matrix containing a drug or combinations of drugs and a covering layer. The upper surface of the backing sheet is coated with an adhesive, which secures the support substrate to the backing sheet. The polymer matrix is applied to the upper surface of the support substrate. The covering layer is placed directly on top of the polymer matrix. The drug may comprise a supersaturated solution of the polymer matrix with a drug or drugs. The support substrate is placed in the center of the backing sheet, and the remaining exposed area of the adhesive will be used to adhere the dressing to the animal skin. The support substrate is added to provide a desirable cushioning effect when the dressing is applied to a wound site.

In an alternative embodiment of the present invention, the dressing does not have any covering layer, and the polymer matrix is placed in direct contact with the skin of the animal. If the dressing has release sheets, the release sheets will cover the polymer matrix layer until the release sheets are removed and the polymer matrix placed against the skin.

In yet another embodiment of the present invention the dressing has a reservoir affixed to the backing sheet. The reservoir may contain the polymer matrix in either a liquid or semi-solid form to enable the polymer matrix to be delivered transdermally through an optimal inert porous membrane interposed between the polymer matrix and the skin. The use of a reservoir has the added value of reducing production costs, and allowing variability in the form and quantity of the polymer matrix.

The dressing of the present invention may also comprise one or more release sheets. Preferably, the dressing will have two release sheets. The release sheets completely cover the exposed adhesive surface of the backing sheet as well as the covering layer. The release sheets are tabbed so that they may be pulled off of the dressing prior to the application of the dressing to the skin.

The backing sheet is preferably a layer of material impervious to both oil and water, such as a synthetic polymer, acetate compound, plastic material, silicone based material, or the like. Ideally, the backing sheet is from about 1 ml to about 10 ml thick.

The backing sheet may also be formed from an inert fluorine-containing addition polymer or from poly(tetrafluoroethylene). The backing sheet must be inert to the polymer matrix while also being permeable or impermeable to oil and water.

The backing sheet may be waterproof. A waterproof dressing would be desirable because it could create a seal around the area to be treated to enable the drug to be absorbed without being washed away. The backing sheet may be any color, and may also have designs or characters on it making the dressing more acceptable to children.

The adhesive that bonds the backing sheet to the support substrate as well as the skin of the animal may be selected from a wide variety of adhesives well known to one of ordinary skill in the art. The adhesive may be pressure sensitive. A particularly preferred adhesive is a medical grade silicone adhesive which will not be solubilized by the polymer matrix.

If the dressing includes a support substrate, the support substrate may be a synthetic or natural woven fabric. The support substrate may also be a non-woven fabric, such as polyester, nylon or a polyester nylon blend. The support substrate may also be a knitted fabric or a foam.

The support substrate may be cut to a size which covers only the area of the skin which is being treated. In another embodiment of the present invention, additional layers of support substrate may be added under the polymer matrix in order to give the dressing a more quilted, comfortable feel.

In one preferred embodiment of the present invention, a covering layer is used to reinforce the polymer matrix for application to the skin of the patient. The fabric of the covering layer should be elastic, or a fibrous or porous sheet material such as cotton or polyester felt, or the like which will allow for good bonding during the transdermal process and also is somewhat elastic in nature. Preferably, the fabric which is used as the covering layer of the present invention is non-woven and porous. More preferably, the covering layer may be a polymer selected from the group consisting of polyvinyl chloride, polyethylene, polypropylene, polyester, nylon and mixtures thereof. Preferably, the covering layer has a percent open area of least 20% but no greater than 88%. When the porous covering layer is placed over the polymer matrix, the drug is pushed into the pores and dispersed throughout the covering layer. The circumference of the covering layer is greater than the circumference of the polymer matrix to allow for the increased diameter of the polymer matrix when pressure or shearing forces are applied to the dressing.

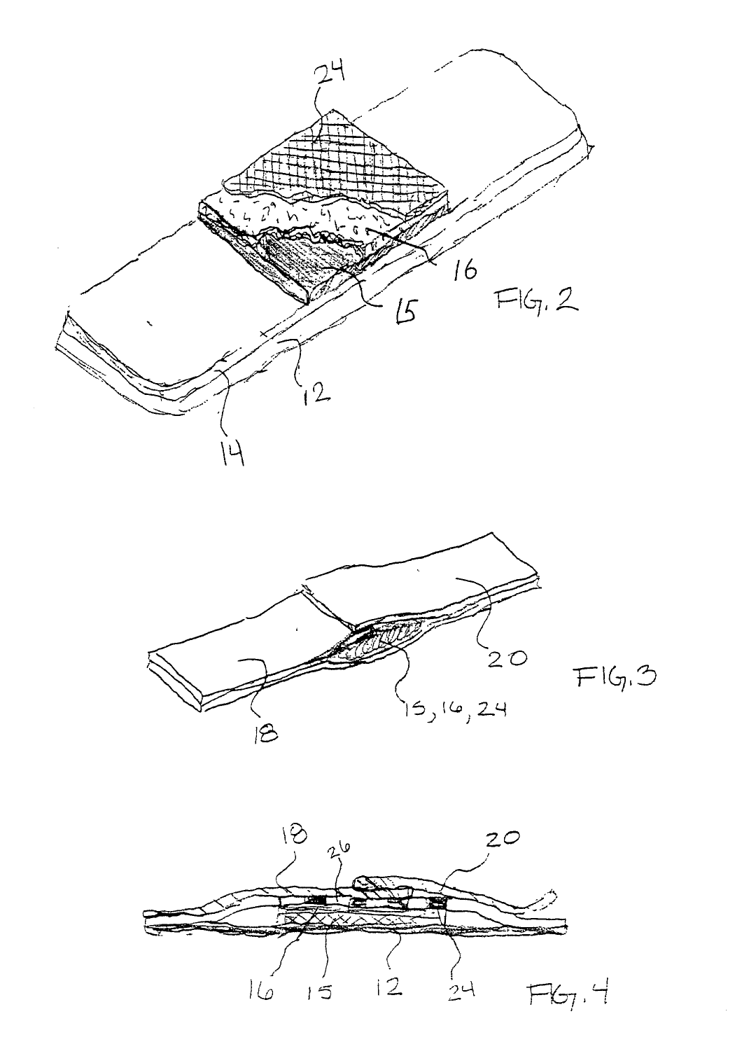

A particularly preferred release sheet is one formed from an inert fluorine-containing addition polymer. The release sheet or sheets should extend beyond the edge of the covering layer, as seen in FIGS. 2 and 3, to provide grasping tabs with which to remove them from the dressing before use.

The polymer matrix of the present invention is created by suspending or solubilizing specialized polymeres in water. At least one of the polymers used to form the matrix must be sufficiently negatively charged to aid in the dispersion, encapsulation, or solubilization of the drug. Particularly preferred polymers have mean average molecular weights below about 800,000 and preferably molecular weights between about 500,000 to 800,000 have been found acceptable to form usable polymer matrixes. Polymers with mean average molecular weights between about 700,000 and 775,000 are most preferred. Polymers having molecular weights above about 800,000 form solid gels in solution and are unable to serve in the present invention. Furthermore, the polymers must be sterilizable and be stable during sterilization so that the polymer does not lose molecular weight once formulated into the final form.

The molar ratio of the polymers present in the matrix is critical. It has been found that molar ratios of the negatively charged polymer to the nonionic polymer must be from 1:0.5 to 4 and preferably from 1:0.5 to 3.0 and most preferably from 1:0.7 to 2.5. At ratios either higher or lower than these levels the resulting systems tend to sheer when being prepared and form unacceptable air pockets and bubbles. Furthermore, the solutions tend to separate and form distinct polymer layers.

Exemplary, non-limiting examples of compounds that may be used as a source of this molecular weight polymer include polysulfated glucosoglycans, glucosaminoglycans, mucopolysaccharides, derivatives thereof, and mixtures thereof. Particularly preferred mucopolysaccharides are chondroitin sulfate and hyaluronic acid salts (sodium or potassium) with sodium hyaluronate being most preferred.

One negatively charged polymer important in the formation of the polymer matrix of the present invention is hyaluronic acid (HA) . Hyaluronic acid (HA) occurs naturally in joint synovial fluid, where it plays a lubricating role, and may have biological activity as well. Because hyaluronic acid possesses a negative charge at neutral pH, it is soluble in water, where it forms highly viscous solutions. A variety of substances, commonly referred to as hyaluronic acid, have been isolated by numerous methods from various tissue sources including umbilical cords, skin, vitreous humor, synovial fluid, tumors, haemolytic streptococci pigskin, rooster combs and the walls of veins and arteries. It may also be synthesized artificially and by recombinant technology.

Conventional methods for obtaining hyaluronic acid results in a product having differing properties and a wide range of viscosities. U.S. Pat. No. 2,585,546 discloses an example of a method for obtaining hyaluronic acid which involves extracting acetone-washed umbilical cords with a dilute salt solution, acidifying the resulting extract, removing the clot so formed, precipitating some hyaluronic acid with protein from the acidified extract with ammonium sulfate, agitating the liquid with pyridine, precipitating another fraction highly contaminated with protein, followed by more ammonium sulfate which forces some pyridine out of solution along with the high viscosity hyaluronic acid. The hyaluronic acid collects at the interface between the two liquid phases and may be separated by filtration, centrifugation, or other usual procedure. A modification of this process involves the fractionation of the acidic salt extract from umbilical cords with alcohol and ammonium sulfate. Alcohol is added to the acidic salt extract, and the resulting precipitate is removed. Solid ammonium sulfate is added to the liquid until saturation and the solution forms two phases with a precipitate of hyaluronic acid at the interface.

One particular fraction of hyaluronic acid that exhibits excellent matrix formation is hyaluronate sodium having an average molecular weight of between 650,000 and 800,000, preferably between 700,000 and 775,000 with a high degree of purity, 95-100% free, and preferably at least 98% pure, from contamination of related mucopolysaccharides. Furthermore, this hyaluronic acid has a sulfated ash content of less than 15% and a protein content of less than 5%. Examples of usable base salts include those safe for animal and human use, such as sodium, potassium, calcium, zinc, and magnesium salts or the like.

In contrast to HA, chondroitins are mucopolysaccharides comprising repeating units of D-glucuronic acid and N-acetyl-D-galactosamine. Chondroitin sulphates are important components of cartilage and bone and are excellent for preparing the polymer matrix herein.

Turning to the actual preparation of the polymer matrix, the first step requires that the negatively charged polymer, such as hyaluronic acid be solubilized. The solution is then allowed to stabilize until a stable solution is formed. Next, a non-ionic polymer such as hydroxyethylcellulose is blended with the hyaluronic solution and allowed to form a polymer matrix. Additional emulsifiers, suspending agents and preservatives may be then added to this system. One particularly nonlimiting effective material for solubilizing water insoluble drugs is methoxypolyethlene glycol (MPEG). At this point, a therapeutically effective amount of a drug or drugs may be added to the matrix.

The negatively charged polymers are generally present in the system in amounts which enable a solution or solid gel to be formed. Generally, solutions are formed using amounts of about 2.0 to about 70.0% by weight with amounts of about 2.3 to about 37.0% by weight being preferred for use with a non-woven fabric sheet. A particularly preferred sodium HA concentration for use with a non-woven fabric sheet is 2.5% by weight.

The nonionic polymer of the polymer matrix, on the other hand, aids in retarding the rate of absorption of the active drug and delays or slows down an animals natural absorption of the negatively charged polymer. Without the presence of this component, the drug would be rapidly absorbed, and sustained action of the active could not be achieved. Particularly preferred nonionic polymers are cellulose derivatives and particularly those selected from the group consisting of carboxymethylcellulose sodium, hydroxyethyl cellulose, hydroxypropyl cellulose, and mixtures thereof.

Additionally, non-ionic polymers have been found to possess exceptional ability to form sustained release matrix formulations when used in combination with the negatively charged polymer. Such polymers are generally employed in amounts of about 0.1% to about 1.0% and preferably about 0.5 to 1.0%. Amounts above about 1.0% result in the formation of a solid gel product if used with the negatively charged polymer. Amounts below about 0.1% have not been found suitable to prepare a storage stable solution or form a product that has sustained drug release.

This combination of negatively charged polymers blended with non-ionic polymers is believed to form a matrix which microencapsulates, suspends, and/or entraps the drug entity such that when it is administered it is slowly released into the systemic circulatory system or muscular tissue providing a sustained and prolonged drug release rate.

A preferred method of making the polymer matrix of the invention will now be described in greater detail.

In order to obtain a hyaluronic acid solution, any conventional method can be used. Sodium hyaluronate or hyaluronic acid from any source can be dissolved in water or in physiological saline to a desired concentration and then a drug is dissolved or dispersed in the resulting solution. More preferably, a sample of HA or a salt of HA is dissolved in water to make an aqueous solution. HA from any variety of sources can be used. The dissolving of the hyaluronic acid in water can occur at any convenient temperature but preferably is conducted between about 15° C. and about 40° C. to provide the first aqueous solution substantially saturated with the hyaluronic acid and its salts.

Preferably, the concentration of HA in this first solution is in the range of about 0.1% to about 5.0% by weight, more preferably in the range of about 0.4% to about 3.5% by weight, and most preferably in the range of about 1.0% to about 3.0% by weight. The precise concentration will vary depending on the molecular weight of the HA.

The hyaluronic acid and its salts useful in the present invention can have widely varying molecular weight but generally have a molecular weight of between about 5,000 daltons and about 8,000,000 daltons, preferably between about 50,000 daltons and about 4,000,000 daltons, and most preferably between about 100,000 daltons and about 1,000,000 daltons.

When highly viscous systems are required the step of concentrating must be practiced under conditions that avoid degradation of the hyaluronic acid and its salts. These conditions can be determined without undue experimentation by a person of ordinary skill in the art. Concentrating is generally practiced until between about 10 percent by weight and about 70 percent by weight, and preferably until between about 20 percent by weight and about 50 percent by weight, and most preferably between 30 percent and 40 percent of the water is removed from the first aqueous solution.

Generally, the concentrated solutions of the present invention may be prepared by slowly adding hyaluronic acid to sterilized water being stirred at approximately 700-1000 rpms. The molecular weight and purity of the hyaluronic acid as described previously are of the utmost importance and must not be significantly changed during processing, therefore mild processing conditions are required. Stirring is continued until the HA has completely dissolved into the water and a crystal clear viscous solution has formed. Next, a quantity of the solution is removed and placed in a clean vessel, where constant stirring is continued. The vessel is then placed in a warm environment, and water content is removed by evaporation, and monitored, without causing the molecular degradation of the HA. The amount of water removal may be determined by the weight reduction of the solution. If weighing the solution does not indicate the desired amount of water either present or removed, the vessel may be returned to the warm environment for further water removal.

The first aqueous solution which is saturated with hyaluronic acid and its salts generally has a maximum concentration of hyaluronic acid and its salts of between about 0.1% and about 10% by weight. Preferably, said solution has a maximum concentration of hyaluronic acid and its salts between about 1% and about 3.5% by weight of hyaluronic acid and its salts based on the weight of the first aqueous solution.

One unique feature of using a polymer matrix is that it can be contoured during manufacture resulting in a matrix of variable thickness and curvature. Similarly, the polymer matrix can be contoured to form a matrix of variable thickness with a central area of zero thickness where an aperture can be created. The matrix can also be of uniform thickness. The thickness of the polymer matrix can be from 0.01 to 1.0 cm, or thicker if desired. The polymer matrix is highly flexible, and can conform to the shape of the skin and surrounding area being treated so as to apply a drug in a prescribed and even manner.

In the polymer matrix of the present invention, hyaluronic acid is preferably present in the form of its salt with a pharmaceutically acceptable cation. Examples of suitable cations include, among others, calcium, magnesium, zinc, and sodium and potassium, wherein sodium is preferred.

According to another aspect of the present invention the composition further comprises a nonionic polymer. While any non-toxic nonionic polymer can be employed, preferably, the nonionic polymer is selected from the group consisting of hydroxyethylcellulose, hydroxypropylcellulose, carboxymethylcellulose, and mixtures thereof. Most preferably, the nonionic polymer is a polymeric cellulose derivative such as hydroxyethylcellulose.

Hyaluronic acid is mucopolysaccharide, and may alternatively be referred to as a glycosaminoglycan. The repeating unit of the hyaluronic acid is a disaccharide consisting of D-glucuronic acid and N-acetyl-D-glucosamine. Because hyaluronic acid possesses a negative charge at neutral pH, it is soluble in water, where it forms a highly viscous solution. The D-glucuronic acid unit and N-actyl-D-glucosamine unit are bonded through a glycosidic, beta (1-3) linkage, while each disaccharide unit is bonded to the next disaccharide unit through a beta (1-5) linkage. The beta (1-4) linkages may be broken through hydrolysis with the enzyme hyaluronidase.

According to another aspect of the present invention, the composition further comprises an effective amount of a therapeutic agent. A wide variety of drugs which are administered may be used in the delivery system according to this invention.

The dressing of the invention is used to treat or prevent drug-induced, alcohol-induced, biologically-induced, trauma-induced or pain-induced nausea, vomiting, dizziness and other adverse effects arising from but not limited to motion sickness, cancer therapy, and pregnancy. Difficulties experienced in adaptation to various forms of travel or movement are also treatable via embodiments of the present invention.

Nausea, dizziness and vomiting in certain cases is caused by excessive stimulation of the vestibular apparatus during motion. While the complete physiological mechanism is not fully understood, it is believed that a combination of visual stimuli, poor ventilation and emotional factors precipitate attacks of motion sickness.

It is generally believed that treating person susceptible to motion sickness prior to onset of symptoms produces a greater reduction in the severity of distress than treatment after symptoms have developed. Utilization of a dermal patch produced with an effective motion sickness medicament applied approximately one to four hours prior to exposure to precipitating factors can deliver an effective and prolonged dosage. If extended exposure to travel is anticipated, a dermal patch produced with a more appropriate dosage amount may be administered.

Due to the complexity and combined nature of the symptoms, a variety of drug treatment options may be employed. In this regard, drugs such as serotonin receptor antagonists, anti-dopaminergics, metclopramides, and scopolamine, dronabinol, ondansetron, granisetron, phenothiazine, thioridazine, diazepam, meclizine, ergoloid mesylates, chlorpromazine, trimethobenzamide, thiethylperazine, perphenazine, hydroxyzine pamoate, compazine, peragen, thorazine, tigan, or mixtures thereof may be employed.

The dressing may also be used to treat or prevent alcohol-related disorders or diseases. Some drugs useful to treat or prevent alcohol related disorders or diseases in combination and within the scope of the present invention include benzodiazepines, barbiturates, librium, serax, tranxene, valium diazepam, lorazepam, oxazepam, and lorazepam, or any mixtures thereof.

It has also unexpectedly been found that when the system is administered in a repetitive manner, once the effects of the active drug are reduced in intensity or effectiveness, such repeat treatments result in a synergistic effect by enhancing the initial term of relief to a period which exceeds the initial time of relief. This is also experienced on subsequent treatments. In this way the present formulations are able to extend relief or treatment from normally several hours to at least 24 hours to several days of relief.

One type of drug that may be used in the present invention are drugs selected from the group of chemotherapeutics such as actinomycin D, adriamycin, altretamine, asparaginase, bleomycin, busulphan, capecitabine, carboplatin, carmustine, chlorambucil, cisplatin, cyclophosphamide, cytarabine, dacarbazine, daunorubicin, doxorubicin, epirubicin, etoposide, fludarabine, fluorouracil, flutamide, gemcitabine, hydroxyurea, idarubicin, ifosfamide, interferon, irinotecan, leuprolide, liposomal doxorubicin, lomustine, megestrol, melphalan, mercaptopurine, methotrexate, mitomycin, mitozantrone, mechlorethamine oxaliplatin, procarbazine, steroids, streptozocin, taxol, taxotere, tamoxifen, tamozolomide, thioguanine, thiotepa, tomudex, topotecan, treosulfan, vinblastine, vincristine, vindesine, vinorelbine, buserelin, chlorotranisene, chromic phosphate, dexamethasone, estradiol, estradiol valerate, estrogens conjugated and esterified, estrone, ethinyl estradiol, floxuridine, goserelin, and prednisone, or mixtures thereof.

Additionally, other drugs that can be used includes drugs selected serotonin receptor antagonists, anti-dopaminergics, metclopramides, and scopolamine, dronabinol, ondansetron, granisetron, phenothiazine, thioridazine, diazepam, meclizine, ergoloid mesylates, chlorpromazine, trimethobenzamide, thiethylperazine, perphenazine, hydroxyzine pamoate, compazine, peragen, thorazine, tigan, or mixtures thereof.

Regardless of the route of administration elected, the formulations of the present invention are formulated into pharmaceutically acceptable dosage forms by conventional methods known in the pharmaceutical art.

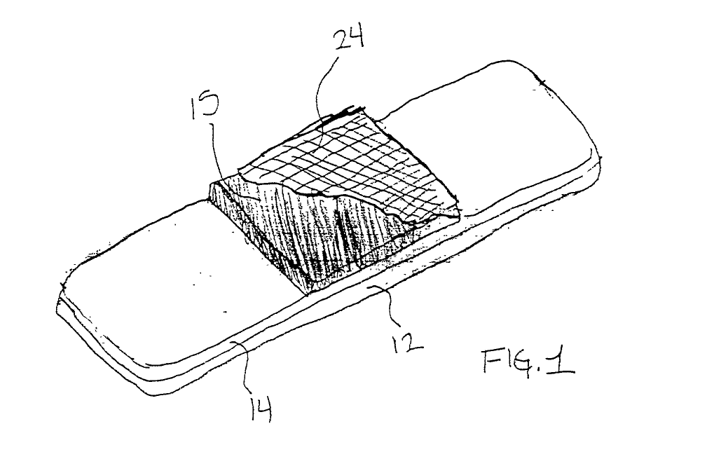

The following is a description of several embodiments of dermal adhesive dressings of the present invention. One particular dressing is illustrated in FIG. 1 of the drawings. The adhesive dressing comprises a backing sheet 12 having apertures therein, a polymer matrix 15 and a porous covering layer 24. The upper surface of the backing sheet was coated with a layer of a pressure sensitive adhesive 14. It will be understood that any of the adhesive well known in the art for use with adhesive bandages may be used in place of this adhesive. The adhesive may, if desired, be deposited on the backing sheet in a continuous or discontinuous pattern rather than as an overall coating, as seen in the drawing.

The upper surface of the backing sheet carries and has adhered thereto a polymer matrix 15. The polymer matrix 15 is centered within the backing sheet and optimally extends from one side of the backing sheet to the other side (see FIG. 1). The upper surface of the polymer matrix 15 is covered by a webbed covering material 24 such as a polyethylene film. Other porous covering materials may be used in place of the aforementioned polyethylene film.

The polymer matrix 15 used in the adhesive dressing of this example contains a supersaturated solution of hyaluronic acid and hydroxyethylcellulose. The webbed covering layer 24 overlies the upper surface of the polymer matrix and is coextensive in length and width with the polymer matrix.

FIG. 2 shows the above dressing with the addition of a support substrate 15. The support substrate 15 is preferably provided in the form of a fibrous pad which is centered from one side of backing sheet 12 to the other side of the backing sheet. It will be understood that support substrate 15 is secured to backing sheet 12 by adhesive layer 14. The polymer matrix 16 overlies support substrate 15. The function of the support substrate is to support the polymer matrix, as well as to provide a desirable cushioning effect when the adhesive dressing is applied. The upper surface of polymer matrix 16 may be covered by a webbed covering layer 24, as discussed above.

FIGS. 3 and 4 show different views of the dressing of FIG. 2 with the release sheets 18, and 20 shown. Release sheets 18, and 20 were placed over the exposed portions of adhesive 14 and the upper surface of webbed covering layer 24 in such as way as to create tabs. The tabs are used to remove the release sheets before administering the dressing.

In addition, FIG. 4 shows pores 26 in webbed covering layer 24. When the dressing is applied to a wound, the pressure will force drug in the polymer matrix up through pores 26, allowing the drug to contact the skin.

FIG. 5 shows a sectional view of the dressing of FIG. 2, allowing a view of all of the layers in said dressing. The upper surface of backing sheet 12 is coated with adhesive layer 14. The support substrate 16 rests upon the adhesive layer. The polymer matrix 15 may lie below or above the support substrate 16 and covering layer 24. Pores 26 in the covering layer 24 are shown. In addition, the figure shows polymer matrix containing the drug 28, which has been forced through pores 26 and is now able to contact the skin when applied.

FIG. 6 is a perspective view of another embodiment of the invention with portions cut away.

The device comprises a laminated composite of adhesive overlay 68, a backing sheet 65 underlying adhesive overlay 68 and a membrane 70 permeable to the polymer matrix contained within reservoir 55. Release sheets 67 cover the adhesive. A support structure 60 may also be included.

The reservoir 55 is affixed to either support structure 60 or backing sheet 65. The reservoir may be affixed by gluing, mechanically fixing or through interlocking means or any other means known to one of ordinary skill. Alternatively, the reservoir 55 may be molded or integrally formed from the material forming the backing sheet or the support structure.

A peel seal disc (not shown) may underlie the permeable membrane and a heat seal (not shown) may be set about the periphery of the peel seal disc. The peel seal disc protects against release of the active agent from the reservoir and the heat seal protects the active agent from exposure to the environment prior to use.

Finally, the permeable membrane may contain apertures 71 to facilitate delivery of the polymer matrix. Alternatively, the membrane may be impermeable with the apertures 71 being the only means of delivery of the polymer matrix, wherein the apertures are configured to control the delivery and release rate of the polymer matrix.

FIG. 7 is a cross-sectional view of the dressing of FIG. 6, showing the reservoir integrally formed or molded with the backing sheet 65.

The adhesive dressing of the present invention can be applied to various portions of the skin of an animal in need of such treatment. A non-limiting list of examples of body parts for which the present adhesive dressing is useful includes the forehead, nose, neck, throat, arm, elbow, wrist, finger, chest, stomach, back, breast, leg, knee, ankle, foot and toe. In order to best fit specific body parts, the dressing of the present invention may be rectangular, as shown in FIG. 1. In additional embodiments of the present invention, the dressing may be circular or butterfly shaped (“H” shaped to best fit around fingers and toes). The dressing of the present invention can be small, large or sized to fit a specific body part.

The adhesive dressing of the present invention may also be in the form of a patch. The patch may be placed against the skin to administer a dosage of drug, alone or in combination with additional drugs to an animal. The patch may be placed anywhere on the body where there is skin. Preferably, the patch may be placed on the back of the neck.

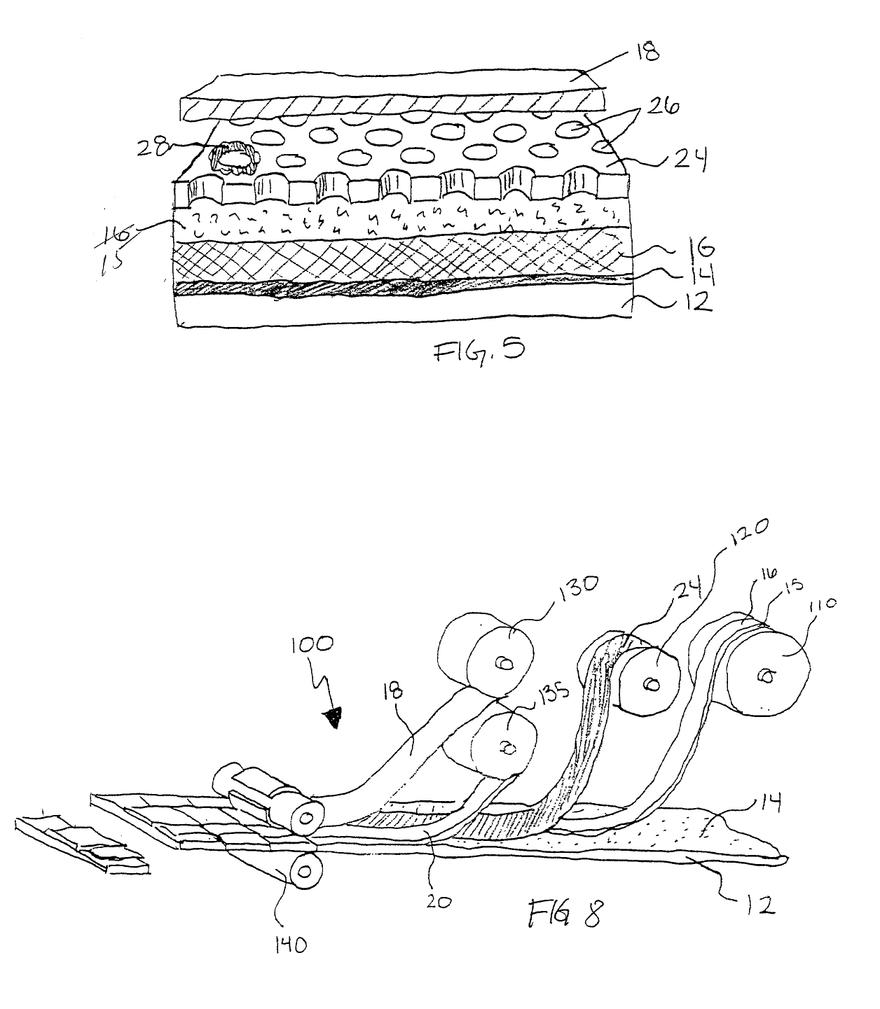

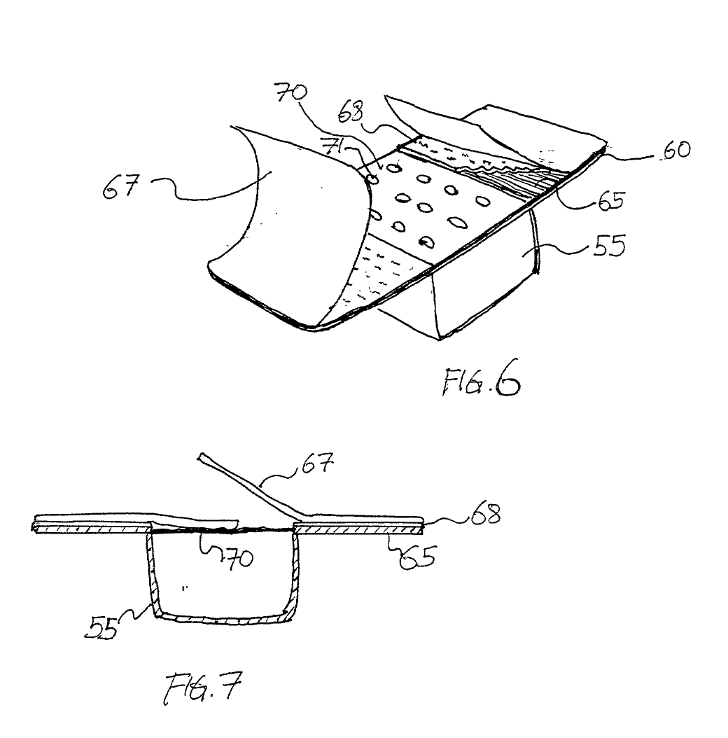

As shown in FIG. 8, the method of manufacturing dermal adhesive dressings requires a machine, a roll for supplying the support substrate, a roll for supplying the covering layer, one or more rolls for supplying release sheets, and two cutter rollers. The upper surface of the support substrate will have previously been coated with the polymer matrix containing the drug.

Next, a backing sheet having indefinite length is provided. The upper surface of the backing sheet will have previously been coated with an adhesive.

The support substrate is led off of the roll and placed on the center of the upper surface of the backing sheet. The covering layer is led off of the roll and placed on the support substrate. The release sheet is led off of the roll and placed on the covering layer and the upper surface of the backing sheet. Finally, the backing sheet, support substrate, covering layer and release sheet are fed through the nip in the cutter rollers to produce a plurality of adhesive dressings.

Specifically, the adhesive dressing may be manufactured according to a process in which the dressing is oriented at right angles to the direction of travel of the raw materials through the manufacturing apparatus. The backing sheet 12 coated with adhesive 14 is conveyed, from right to left, on top of a conveyor belt (not pictured).

As shown in FIG. 8, a web comprising the support substrate 15 onto which the polymer matrix 16 has been previously applied by an extrusion coating process is led off of the roll 110 and placed on top of the adhesive 14 coated backing sheet 12. The width of the web corresponds to the length of the backing sheet.

The covering layer 24 is led off a supply roll 120 and placed on top of the web. It will be understood that in the process being described, the width of the covering layer corresponds substantially to the width of the web. Release sheets 18, 20, taken from rolls 130, 135, are applied so as to cover the exposed adhesive area at the other side of the adhesive coated backing sheet 12 as well as the upper surface of covering layer 24. Release sheets 18, 20 extend beyond the edge of the covering layer 24 to provide grasping tabs.

The combined raw materials, as described above, are then passed through the nip of cutter rollers 140. The rollers compress the raw materials at a pressure of about 10-20 pounds per square inch and, at the same time, cut the traveling raw materials into individual adhesive dressings. As a result of the described process, the polymer matrix 16 is pressed up into holes 26, 28 in the covering layer 24 so that the polymer matrix is in intimate contact with the lower surfaces of release sheets 18, 20, as shown in FIG. 4. The individual adhesive dressings are subsequently wrapped, sterilized and packaged according to procedures which are well known in the art.

After the release sheets are removed prior to use and the adhesive dressing is applied over the wound site, the surface of the wound is contacted by portions of the polymer matrix which had previously been in contact with the release sheets.

The dermal adhesive dressing is placed on the animal skin (not shown) such that the polymer matrix containing the drug is in contact with the skin. The dressing may be applied to the skin such that the drug is administered to the skin over a long period of time.

Dosing Amounts

As discussed above, an effective but nontoxic amount of the system is employed in treatment. The dose regimen for administering drugs or treating various conditions, such as nausea as described above, is selected in accordance with a variety of factors including the type, age, weight, sex, and medical condition of the subject, the severity of the pain, the route of administration and the particular complex or combination of drugs employed. Determination of the proper dose for a particular situation is within the skill of the art. Generally, treatment is initiated with smaller dosages which are less than the optimum doses of the compound. Thereafter, the dose is increased if necessary by small increments until the optimum effect under the circumstances is reached. For convenience, the total daily dosage may be divided and administered in portions during the day if desired. Generally, amounts of matrix with drug may vary from 0.0001% to about 75% by weight of the system when used topically with 2 to 25 mg concentrations and preferably in 3 to 10 mg amounts.

It should be further noted that a significant advantage of the dosage form of the present system relates to its ability to allow the drug to slowly diffuse through tissue when administered, thus allowing for an effective therapeutic dose to be present for many hours. In this regard, it should be noted that reference to a therapeutically effective dose does not necessarily relate to conventional dosage levels, but does relate to drug levels that achieve an effective therapeutic level at the dose employed, which may be the same level but not at the same frequency of administration previously required for drugs taken orally or by injection. This not only significantly reduces the number of doses required to achieve the same effect, but it also reduces costs, maintenance and health hazards associated with conventional treatment therapies. Additionally, it results in immediate and continued drug release for long periods of time spanning several hours in duration.

Doses may vary from patient to patient depending on the type and severity of the condition being treated and the drug being administered. Generally, doses of 1 ml to 75 mg may be administered with preferred doses using 2 to 25mg of the gelled matrix system.

Examples of dosing

| | |
|---|---|
| Ingredient | Quantity (grams) |

| | | |
|---|---|---|
| Marinol | 5.0 | mg |
| Zofran | 4.0 | mg |
| Kytil | 1.0 | mg |
| Promethazine | 26 | mg |
| prochloroperazine | 10 | mg |
| Valium | 2.0-10 | mg |
| Tigan | 100 | mg |
| Torecan | 10-30 | mg |
| Trilafon | 8.0-16 | mg |
| Vistaril | 25-100 | mg |
| chlorodiazepoxide | 50-100 | mg |
| Diazepam | 5.0 | mg |
| dimenhydrinate | 50-100 | mg |

Concentration Potentials

One useful aspect of the present invention is that the matrix may be concentrated to various degrees depending upon intended usage. The step of concentrating, must be practiced under conditions that avoid degradation of the hyaluronic acid and its salts, non-ionic polymer component and the polymer matrix. These conditions can be determined without undue experimentation by a person of ordinary skill in the art. Concentrating is generally practiced until between about 10 percent by weight and about 70 percent by weight, and preferably until between about 20 percent by weight and about 55 percent by weight, and most preferably between 30 percent by weight and 40 percent by weight of the water is removed from the polymer matrix. Concentrating polymer matrix results in advantageous viscoelastic and Theological properties.

A number of techniques may be employed to dehydrate the solution ranging from the use of solvents and rotary evaporation to heating a previously prepared hyaluronic acid salt solution or polymer matrix. Preferably, the water removal step is affected by controlling the temperature of the solution. Widely varying temperatures can be employed for the concentrating step, however, the temperature is generally maintained from about 10° C. to about 80° C. and preferably from about 30° C. to about 60° C. Subatmospheric pressure may also be used. While, superatmospheric pressure is suitable, this step is preferably practiced at atmospheric pressure, namely about 760 mmHg.

Another method of concentrating the polymer matrix is by supersaturating the hyaluronic solution prior to blending with a non-ionic polymer. For example, the polymers may be dissolved in water and purified either separately or jointly and then the optional active drug added to the system. Again, 10% to about 70% of the water may be removed from the solution, with a preferred range of 37%. When 37% of the water is removed, preferably, the supersaturated solution of hyaluronic acid is present in the polymer matrix in an amount from about 37% to about 40.1% by weight. More preferably, the supersaturated solution of hyaluronic acid is present in the polymer matrix in an amount from about 37.2% to about 39.2% by weight. Even more preferably, the supersaturated solution of hyaluronic acid is present in the polymer matrix in an amount from about 37.6% to about 38.9% by weight. Any conventional means may be used to de-hydrate the hyaluronic acid solution or polymer matrix including but not limited to the use of heat, solvents or rotary evaporation. Generally, the concentrated solutions of the present invention may be prepared by slowly adding hyaluronic acid to sterilized water while stirring at approximately 700-1000 rpms.

It should be noted that the molecular weight and purity of the hyaluronic acid as described previously are of upmost importance and must not be significantly changed during processing. Therefore mild processing conditions are required. Stirring is continued until the HA has completely dissolved into the water and a crystal clear viscous solution has formed.

Next, a quantity of the solution is removed and placed in a clean vessel, where constant stirring is continued. The vessel is then placed in a warm environment and is monitored. The water content is removed by evaporation without causing the molecular degradation of the HA. The amount of water removal may be determined by the weight reduction of the solution. If weighing the solution does not indicate the desired amount of water either present or removed, the vessel may be returned to the warm environment for further water removal.

According to another aspect of the present invention the composition further comprises an active therapeutic agent. Any active therapeutic agent which is compatible the polymer matrix is employed in the present invention. A wide variety of medicaments which are administered may be used in the delivery system according to this invention. Several methods of concentrating the polymer matrix either prior to the addition of a therapeutically effective amount of drug or before may be used.

A particularly preferred concentrating procedure involves separately dissolving the nonionic polymer in water and centrifuging the material to form a solution and then removing impurities. This may be conveniently done at rotation speeds of 2000 rpm for times of about 30 minutes to about two hours.

Another concentration process requires that the negative charged polymer be blended and stirred in water until it is dissolved. This process must be done while avoiding the formation of bubbles and while freeing the polymer of its electrostatic activity. Furthermore, the molecular weight of the polymer must not be significantly changed during processing and as such mild process conditions are required. Processing conditions of 400-3000 rpm for durations of 16-24 hours have been found acceptable to produce stable solutions or gels of the charged polymer.

Conventional pharmaceutically acceptable emulsifiers, suspending agents, antioxidant (such as sodium meta-bisulfate) and preservatives (such as benzyl alcohol) may then be added to this system. Once all the components are blended together, such as by mixing 400-3000 rpm for one to four hours, the system is filled into tubes and sterilized. The resulting system is a clear gel which is storage stable for several years.

The drug may then be added to the homogenous solution or gel separately once dissolved or disbursed in water. Additional emulsifiers, suspending agents and preservatives may be then added to this system. One particularly nonlimiting effective material for solubilizing water insoluble drugs is methoxypolyethlene glycol (MPEG). Once all the components are blended together, for 400-3000 rpm for 1 to 4 hours, the system is filled into tubes and sterilized. The resulting system is storage stable for several years.

The formulations may then be used topically and also contain conventional pharmaceutical acceptable excipients well known to those skilled in the art, such as surfactants, suspending agents, emulsifiers osmotic enhancers, extenders and dilutants, pH modifiers as well as fragrances, colors, flavors and other additives. When used in the dressing as disclosed herein, the pressure of the webbed layer ensures that the polymer matrix remains in contact with the animal skin.

As indicated above, the active drug agents may be blended with the aqueous polymer matrix at the time of manufacture. As such, the drug when in the form of a water-soluble solid is simply diluted with sterilized water or polymer matrix solution and prepared in gel form.

### EXAMPLES

The following examples are illustrative of preferred embodiments of the invention and are not to be construed as limiting the invention thereto. All polymer molecular weights are mean average molecular weights (and represent dalton numbers). The following example illustrates a method of making the polymer matrix used in the dressing of the present invention. All percentages are based on the percent by weight of the final delivery system of the formulation prepared unless otherwise indicated and all totals equal 100% by weight of the product formed.

### Example 1

This example illustrates the synthesis of a hyaluronic acid composition.

The following ingredients are combined as indicated.

| Ingredient | Quantity (grams) |
|---|---|
| Hyaluronate Sodium (HA) | 13.7 |
| Sterile Water | 900 |

Into a sterilized glass vessel is added 500 ml of the sterile water which is stirred at 400-600 rpms. Slowly add 13.7 grams of HA having an average molecular weight of around 700,000 to 775,000.

Allow to stir for 10 to 20 hours until all the HA has dissolved into the water and a crystal clear viscous solution has formed.

A quantity (500 grams) of the above viscous solution is placed in a clean beaker of known weight. A magnetic stirrer of known weight is placed in the beaker. The beaker containing the viscous solution and the stirrer is placed in a laboratory hood where the beaker and its contents are maintained in a warm location at 40° C. while being constantly stirred. Under these conditions water is removed from the viscous solution without any molecular degradation of the HA. At the end of one hour the beaker is weighed. If the weight reduction does not indicate removal of the desired amount of water, the beaker, with its contents, is returned to the warm location in the hood for further water removal.

In this example removal of 37 weight percent of the water is deemed sufficient to prepare a semi-solid material.

### Example 2

This example illustrates the synthesis of a composition of the present invention employing hydroxyethyl cellulose (HEC) as a nonionic polymer in the polymer matrix.

The following ingredients are combined as indicated.

| Ingredient | Quantity (grams) |
|---|---|
| Hydroxyethyl cellulose (HEC) | 12.5 |
| Hyaluronate Sodium (HA) | 13.7 |
| Sterile Water | 900 |

Into a sterilized glass vessel is added 500 ml of the sterile water which is stirred at 400-600 rpms. Slowly add 13.7 grams of HA having a molecular weight of around 700,000 to 775,000 and a purity described previously.

Allow to stir for 10 to 20 hours until all the HA has dissolved into the water and a crystal clear viscous solution has formed.

Prepare a 1.25% solution of HEC by adding 12.5 grams of the solid material under aseptic conditions to 275 ml of sterile water. Allow to dissolve for 1 to 2 hours while stirring thereby forming an HEC solution. Add the HEC solution to the HA solution and mix for 2 to 4 hours at 490 to 600 rpm until a homogenous clear viscous solution which is free of air bubbles is produced.

A quantity (500 grams) of the above viscous solution is placed in a clean beaker of known weight. A magnetic stirrer of known weight is placed in the beaker. The beaker containing the viscous solution and the stirrer is placed in a laboratory hood where the beaker and its contents are maintained in a warm location at 40° C. while being constantly stirred. Under these conditions water is removed from the viscous solution without any molecular degradation of the HA. At the end of one hour the beaker is weighed. If the weight reduction does not indicate removal of the desired amount of water, the beaker, with its contents, is returned to the warm location in the hood for further water removal.

According to another aspect of the present invention the composition further comprises an active therapeutic agent. Any active therapeutic agent which is compatible with hyaluronic acid and its salts can be employed in the present invention. A wide variety of medicaments which are administered may be used in the delivery system according to this invention.

### Example 3

This example demonstrates the formation of a transdermal anti-emetic, anti-motion preparation with dimenhydrinate which is useful for preventing and treating nausea, vomiting, dizziness and other adverse effects arising from but not limited to motion sickness, cancer therapy, and pregnancy in an animal

The present example also demonstrates the formation of a transdermal preparation containing the anti-emetic, anti-motion preparation when administered topically. The onset of this beneficial effect in the form of treating vomiting, nausea, and dizziness occurs between 10 and 20 minutes following topical administration and lasts for up to 6 hours.

The dosage range for the drug is between 1-5 mg.

| Ingredient | Quantity (grams) |
|---|---|
| dimenhydrinate | 1.5% |
| Sodium hyaluronate (HA) | 2.3% |
| Hyroxyethyl cellulose (HEC) | 0.7% |
| Methoxypolyethylene glycol (MPEG) | 10% |
| Benzyl alcohol | 2.5% |
| Water | Remainder |

BATCH SIZE 1500 ml

Into a sterilized glass vessel is added 1062.5 ml of sterile water which is stirred at 1500 to 2000 rpm. Slowly add 34.5 grams of HA, having a molecular weight of around 700,000 to 775,000 and a purity described above. Allow to stir for 16 to 20 hours until all of the HA polymer has dissolved into the water and a crystal-clear viscous solution has formed.

Prepare a 0.7% solution of HEC by adding 10.5 grams of the solid material under aseptic conditions to 250 ml of sterile water. Allow to dissolve for 1 to 2 hours while stirring at 1500 to 2000 rpm. Add the HEC solution to the HA solution and mix for 10 to 15 hours until a homogeneous solution is produced.

Carefully measure 150 ml of methoxypolyethylene glycol (MPEG) 10% into the mixture. RPM speeds should be increased for the mixture while this step is being performed to 2500 rpm. The resulting mixture thus formed should be allowed to mix at 2000 rpm for an additional 3 to 4 hours.

At this point 2.5% of benzol alcohol or 37.5 ml is added to the mixture. Again, the rpm speed is increased during this part of the procedure to 2500. The mixture should be allowed to mix for 3 to 5 hours at 2000 rpm.

Using safe techniques, 25 grams (1.5%) of the diclofenac should be slowly added to the mixture. Again the rpm speed for the purpose of addition of dimenhydrinate should be increased to 2500, and the entire 25 grams of dimenhydrinate should be completed within 15 minutes.

The final mixture is clear with a slight green tint following 15 to 20 hours of further mixing at 2000 rpm. The final product should be transferred, using aseptic technics, to 25 ml borasylicate glass jars with a lined cap.

### Example 4

The formula and method of manufacture of Example 3 are repeated for dimenhydrinate. The only difference is that MPEG is not used.

| Ingredient | Quantity (grams) |
|---|---|
| dimenhydrinate | 1.5% |
| Sodium hyaluronate (HA) | 2.3% |
| Hydroxyethyl cellulose (HEC) | 0.7% |
| Benzyl alcohol | 2.5% |

BATCH SIZE 1500 ml

Into a sterilized glass vessel is added 1062.5 ml of sterile water which is stirred at 1500 to 2000 rpm. Slowly add 34.5 grams of HA, having a molecular weight of around 700,000 to 775,000 and a purity described previously. Allow to stir for 16 to 20 hours until all of the HA polymer has dissolved into the water and a crystal-clear viscous solution has formed.

Prepare a 0.7% of HEC by adding 10.5 grams of the solid material under aseptic conditions to 250 ml of sterile water. Allow to dissolve for 1 to 2 hours while stirring at 1500 to 2000 rpm. Add the HEC solution to the HA solution and mix for 10 to 15 hours until a homogeneous solution is produced.

At this point 2.5 % of benzol alcohol or 37.5 ml is added to the mixture. Again, the rpm speed is increased during this part of the procedure to 2500. The mixture should be allowed to mix for 3 to 5 hours at 2000 rpm.

As described above, using safe techniques, 25 grams (1.5%) of the dimenhydrinate is slowly added to the mixture. Again the rpm speed for the purpose of addition of dimenhydrinate should be increased to 2500, and the entire 25 grams of dimenhydrinate should be completed within 15 minutes.

The final mixture is clear with a slight green tint following 15 to 20 hours of further mixing at 2000 rpm. The final product should be transferred, using aseptic technic, to 25 ml borasylicate glass jars with a lined cap

The invention being thus described, it will be obvious that the same may be varied in many ways. Such variations are not to be regarded as a departure from the spirit scope of the invention and all such modifications are intended to be included within the scope of the following claims.

What is claimed is:

1. A stable, sterilized, purified composition, comprising:
a polymer matrix; and
a therapeutically effective amount of a drug, wherein said drug is used to prevent or treat drug-induced, alcohol-induced, biologically-induced, trauma-induced or pain-induced nausea, vomiting, dizziness and other adverse effects arising from but not limited to motion sickness, cancer therapy, and pregnancy.

2. The composition of claim 1, wherein said drug can be selected from the group of serotonin receptor antagonists, anti-dopaminergics, metclopramides, and scopolamine, dronabinol, ondansetron, granisetron, phenothiazine, thioridazine, diazepam, meclizine, ergoloid mesylates, chlorpromazine, trimethobenzamide, thiethylperazine, perphenazine, hydroxyzine pamoate, compazine, peragen, thorazine, tigan, or mixtures thereof.

3. The composition of claim 1, wherein said drug can be dispersed within said polymer matrix, and where said drug is selected from the group of chemotherapeutics such as actinomycin D, adriamycin, altretamine, asparaginase, bleomycin, busulphan, capecitabine, carboplatin, carmustine, chlorambucil, cisplatin, cyclophosphamide, cytarabine, dacarbazine, daunorubicin, doxorubicin, epirubicin, etoposide, fludarabine, fluorouracil, flutamide, gemcitabine, hydroxyurea, idarubicin, ifosfamide, interferon, irinotecan, leuprolide, liposomal doxorubicin, lomustine, megestrol, melphalan, mercaptopurine, methotrexate, mitomycin, mitozantrone, mechlorethamine oxaliplatin, procarbazine, steroids, streptozocin, taxol, taxotere, tamoxifen, tamozolomide, thioguanine, thiotepa, tomudex, topotecan, treosulfan, vinblastine, vincristine, vindesine, vinorelbine, buserelin, chlorotranisene, chromic phosphate, dexamethasone, estradiol, estradiol valerate, estrogens conjugated and esterified, estrone, ethinyl estradiol, floxuridine, goserelin, and prednisone, or mixtures thereof.

4. The composition of claim 1, wherein said drug can be selected from the group of compounds useful for treating alcohol related disorders or diseases such as benzodiazepines, barbiturates, librium, serax, tranxene, valium diazepam, lorazepam, oxazepam, and lorazepam, or mixtures thereof.

5. The composition of claim 1 wherein said polymer matrix contains a negatively charged polymer in combination with a nonionic polymer.

6. The polymer matrix of claim 5, wherein a molar ratio of negatively charged polymer to non-ionic polymer is 1:0.5 to 4.0.

7. The polymer matrix of claim 5, wherein a molar ratio of negatively charged polymer to non-ionic polymer is 1:0.5 to 3.0.

8. The polymer matrix of claim 5, wherein a molar ratio of negatively charged polymer to non-ionic polymer is 1:0.7 to 2.5.

9. The polymer matrix of claim 5, wherein said nonionic polymer is selected from the group consisting of hydroxyethylcellulose, hydroxypropylcellulose, or carboxymethylcellulose.

10. The polymer matrix of claim 5, wherein said negatively charged polymer is selected from the group of hyaluronic acid, a sodium or potassium salt of hyaluronic acid and mixtures thereof.

11. The polymer matrix of claim 1, wherein said composition is capable of continuously releasing therapeutically effective amounts of said drug over about 1 hour to about 24 hours of time when administered to an animal.

12. The composition of claim 1, wherein the polymer matrix is a formable, flexible, movable sheet.

13. A dermal dressing, comprising:
a polymer matrix containing a negatively charged polymer in combination with a nonionic polymer, wherein the polymer matrix is conformable to topical application on animal skin; and
wherein said polymer matrix contains a therapeutically effective amount of a drug to prevent or treat drug-induced, alcohol-induced, biologically-induced, trauma-induced or pain-induced nausea, vomiting, dizziness and other adverse effects arising from but not limited to motion sickness, cancer therapy, and pregnancy.

14. The dermal dressing of claim 13, wherein said drug can be selected from the group of serotonin receptor antagonists, anti-dopaminergics, metclopramides, and scopolamine, dronabinol, ondansetron, granisetron, phenothiazine, thioridazine, diazepam, meclizine, ergoloid mesylates, chlorpromazine, trimethobenzamide, thiethylperazine, perphenazine, hydroxyzine pamoate, compazine, peragen, thorazine, tigan or mixtures thereof.

15. The dermal dressing of claim 13, wherein said drug can be dispersed within said polymer matrix, and where said drug is selected from the group of chemotherapeutics such as actinomycin D, adriamycin, altretamine, asparaginase, bleomycin, busulphan, capecitabine, carboplatin, carmustine, chlorambucil, cisplatin, cyclophosphamide, cytarabine, dacarbazine, daunorubicin, doxorubicin, epirubicin, etoposide, fludarabine, fluorouracil, flutamide, gemcitabine, hydroxyurea, idarubicin, ifosfamide, interferon, irinotecan, leuprolide, liposomal doxorubicin, lomustine, megestrol, melphalan, mercaptopurine, methotrexate, mitomycin, mitozantrone, mechlorethamine oxaliplatin, procarbazine, steroids, streptozocin, taxol, taxotere, tamoxifen, tamozolomide, thioguanine, thiotepa, tomudex, topotecan, treosulfan, vinblastine, vincristine, vindesine, vinorelbine, buserelin, chlorotranisene, chromic phosphate, dexamethasone, estradiol, estradiol valerate, estrogens conjugated and esterified, estrone, ethinyl estradiol, floxuridine, goserelin, and prednisone, or mixtures thereof.

16. The dermal dressing of claim 13, wherein said drug can be selected from the group of compounds useful for treating alcohol related disorders or diseases such as benzodiazepines, barbiturates, librium, serax, tranxene, valium diazepam, lorazepam, oxazepam, and lorazepam, or mixtures thereof.

17. The dermal dressing of claim 13, wherein said nonionic polymer is selected from the group consisting of hydroxyethylcellulose, hydroxypropylcellulose, or carboxymethylcellulose.

18. The dermal dressing of claim 13, wherein said negatively charged polymer is selected from the group of a hyaluronic acid, a hyaluronic acid salt and mixtures thereof.

19. The dermal dressing of claim 13, wherein the negatively charged polymer is a solution of hyaluronic acid present in an amount of about 37% to about 40.1% by weight.

20. The dermal dressing of claim 13, wherein the negatively charged polymer is a supersaturated solution of hyaluronic acid present in an amount of about 37.2% to about 39.2% by weight.

21. The dermal dressing of claim 13, wherein the negatively charged polymer is a supersaturated solution of hyaluronic acid present in an amount of 36.7% to about 38.9% by weight.

22. The dermal dressing of claim 13, further comprising:
a backing sheet having applied thereto an adhesive capable of securing the dermal dressing to the animal skin;
a reservoir affixed to said backing sheet containing said polymer matrix; and
an inert porous membrane interposed between said polymer matrix and said animal skin.

23. The dermal dressing of claim 22, wherein the dermal dressing has a perimeter edge defining a circumference, a rectilinear perimeter, a triangular perimeter or a perimeter of any geometric shape.

24. The dermal dressing of claim 22, wherein the inert porous membrane has a delivery rate regulating means for dosing the drug over a period of time.

25. The dermal dressing of claim 24, wherein the delivery rate of the porous membrane is about 1 hour to about 24 hours per dose.

26. The dermal dressing of claim 13, further comprising:
a backing sheet overlying said polymer matrix, wherein the backing sheet having applied thereto an adhesive capable of securing the polymer matrix to the backing sheet and the backing sheet to the animal skin; and
a webbed covering layer underlying said polymer matrix.

27. The dermal dressing of claim 13, further comprising:
a covering layer overlying the polymer matrix;
one or more release sheets, wherein said backing sheet having applied thereto an adhesive which secures said support substrate to the backing sheet and the backing sheet to the animal skin;
wherein the release sheets completely cover the adhesive on the backing sheet and the covering layer; and
wherein the release sheets may be peeled off of said adhesive.

28. The dressing of claim 22, wherein the backing sheet is permeable to oil or water.

29. The dressing of claim 22, wherein the backing sheet is impermeable to oil or water.

30. The dressing of claim 22, wherein the backing sheet is inert to hyaluronic acid and its salts.

31. The dressing of claim 22, wherein the webbed covering layer is a natural polymer.

32. The dressing of claim 22, wherein the webbed covering layer is a synthetic polymer.

33. The dressing of claim 32, wherein the synthetic polymer is selected from the group consisting of polyvinyl chloride, polyethylene, polypropylene, polyester and nylon.

34. The dressing of claim 22, wherein the webbed covering layer is sufficiently porous to enable the polymer matrix to contact the skin.

35. A method for administering a drug to an animal, comprising the step of:
applying a dermal dressing to animal skin, wherein the dermal dressing is comprised of:
a polymer matrix containing a negatively charged polymer in combination with a nonionic polymer, wherein the polymer matrix is conformable to topical application on animal skin; and
wherein said polymer matrix contains a therapeutically effective amount of a drug to prevent or treat drug-induced, alcohol-induced, biologically-induced, trauma-induced or pain-induced nausea, vomiting, dizziness and other adverse effects arising from but not limited to motion sickness, cancer therapy, and pregnancy.

36. A method for preventing or treating a condition in an animal comprising the steps of:
applying a polymer matrix film onto the animal on an area to be treated, wherein the polymer matrix film contains a negatively charged polymer in combination with a nonionic polymer, and is formable, flexible and moveable; and
securing said polymer matrix film onto the area to be treated with a dressing fixative.

37. The method of claim 36, wherein the dressing fixative is a bandage selected from the group consisting of a single sided adhesive bandage, a gauze wrap, a stretchable woven wrap and a stretchable sleeve.

38. The method of claim 36, wherein said polymer matrix contains a drug used to prevent or treat drug-induced, alcohol-induced, biologically-induced, trauma-induced or pain-induced nausea, vomiting, dizziness and other adverse effects arising from but not limited to motion sickness, cancer therapy, and pregnancy.

39. The method of claim 36, wherein the polymer matrix film delivers a therapeutically effective amount of a drug upon the animal for about 1 hour to about 24 hours of time.

40. A method for preventing or treating nausea in an animal for a sustained period of time, comprising the step of:
applying to said animal a polymer matrix, comprising a negatively charged polymer and a nonionic polymer in combination with a therapeutically effective amount of a drug for preventing or treating nausea.

41. The method of claim 40, wherein said drug can be selected from the group of serotonin receptor antagonists, anti-dopaminergics, metclopramides, and scopolamine, dronabinol, ondansetron, granisetron, phenothiazine, thioridazine, diazepam, meclizine, ergoloid mesylates, chlorpromazine, trimethobenzamide, thiethylperazine, perphenazine, hydroxyzine pamoate, compazine, peragen, thorazine, tigan, or mixtures thereof.

42. A method for preventing or treating dizziness in an animal, comprising the step of:
applying to said animal a polymer matrix, comprising a negatively charged polymer and a nonionic polymer in combination with a therapeutically effective amount of a drug for preventing or treating dizziness.

43. A method for preventing or treating vomiting in an animal, comprising the step of:
applying to said animal a polymer matrix, comprising a negatively charged polymer and a nonionic polymer in combination with a therapeutically effective amount of a drug for preventing or treating vomiting.

44. A method for preventing or treating pre-operative or post-operative vomiting, nausea or dizziness in an animal, comprising the step of:
applying to said animal a polymer matrix, comprising a negatively charged polymer and a nonionic polymer in combination with a therapeutically effective dose of a drug for preventing or treating vomiting, nausea or dizziness in pre-operative or post-operative procedures.

45. A method for preventing or treating cancer in an animal, comprising the step of:
applying to said animal a polymer matrix, comprising a negatively charged polymer and a nonionic polymer in combination with a therapeutically effective amount of a drug for preventing or treating cancer.

46. The method of claim 45, wherein said drug can be dispersed within said polymer matrix, and where said drug is selected from the group of chemotherapeutics such as actinomycin D, adriamycin, altretamine, asparaginase, bleomycin, busulphan, capecitabine, carboplatin, carmustine, chlorambucil, cisplatin, cyclophosphamide, cytarabine, dacarbazine, daunorubicin, doxorubicin, epirubicin, etoposide, fludarabine, fluorouracil, flutamide, gemcitabine, hydroxyurea, idarubicin, ifosfamide, interferon, irinotecan, leuprolide, liposomal doxorubicin, lomustine, megestrol, melphalan, mercaptopurine, methotrexate, mitomycin, mitozantrone, mechlorethamine oxaliplatin, procarbazine, steroids, streptozocin, taxol, taxotere, tamoxifen, tamozolomide, thioguanine, thiotepa, tomudex, topotecan, treosulfan, vinblastine, vincristine, vindesine, vinorelbine, buserelin, chlorotranisene, chromic phosphate, dexamethasone, estradiol, estradiol valerate, estrogens conjugated and esterified, estrone, ethinyl estradiol, floxuridine, goserelin, and prednisone, or mixtures thereof.

47. A method for preventing or treating alcohol related disorders or diseases in an animal, comprising the step of:
applying to said animal a polymer matrix, comprising a negatively charged polymer and a nonionic polymer in combination with a therapeutically effective dose of a drug for preventing or treating alcohol-related disorders.

48. The method of claim 47, wherein said drug can be selected from the group of compounds useful for treating alcohol related disorders or diseases such as benzodiazepines, barbiturates, librium, serax, tranxene, valium diazepam, lorazepam, oxazepam, and lorazepam, or mixtures thereof.
